(19) United States
(12) Patent Application Publication
(10) Pub. No.: US 2018/0114288 A1
(43) Pub. Date: Apr. 26, 2018

(54) SYSTEM AND METHODS OF IMPROVED HUMAN MACHINE INTERFACE FOR DATA ENTRY INTO ELECTRONIC HEALTH RECORDS

(71) Applicants: Gabriel ALDAZ, Palo Alto, CA (US); Alan E. SHLUZAS, San Carlos, CA (US); David PICKHAM, Redwood City, CA (US); Lauren M. SHLUZAS, San Carlos, CA (US)

(72) Inventors: Gabriel ALDAZ, Palo Alto, CA (US); Alan E. SHLUZAS, San Carlos, CA (US); David PICKHAM, Redwood City, CA (US); Lauren M. SHLUZAS, San Carlos, CA (US)

(21) Appl. No.: 15/795,035

(22) Filed: Oct. 26, 2017

Related U.S. Application Data
(60) Provisional application No. 62/412,844, filed on Oct. 26, 2016.

(51) Int. Cl. G06Q 50/24; G06F 19/00; G02B 27/01; A61B 5/00; H04L 29/08

(52) U.S. Cl. CPC G06Q 50/24; G06F 19/3418; G02B 27/0172; H04L 67/12; G06F 19/323; A61B 5/0002; G06F 19/327; A61B 5/681

(57) ABSTRACT

This disclosure provides an efficient, hands-free system and method for capturing and recording patient data in critical care environments. The systems and methods described herein enables clinicians to record and transcribe patient information onto a disposable medical record tag (akin to a military ID tags), which accompanies the patient throughout initial stabilization and presentation to a treatment center. A Pre-Hospital Treatment & Triage (PHT) guidance system, visible in the HMD, can guide caregivers and/or first responders through treating the patient and documenting a patient's medical condition and treatment status, and triaging patients to the appropriate level of care. The head-mounted display can wirelessly transfer the patient's treatment data to a lightweight disposable data tag, referred to as an electronic TCCC (E-TC3) that is affixed to the patient. The data tag digitally stores a patient's health status, and displays a specific color based on a patient's degree of injury.

### CROSS REFERENCE TO RELATED APPLICATIONS

This application claims the benefit of U.S. Provisional Patent Application No. 62/412,844, filed Oct. 26, 2016, titled “System and Methods of Improved Machine Interface for Data Entry into Electronic Health Records”, the contents of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates generally to the documentation of medical treatment of a patient by a care provider in an electronic medium via data input systems and methods utilizing voice to text software and gesture based input commands.

### BACKGROUND

The Electronic Health Record (EHR) has revolutionized the health environment, providing near real-time documentation and immediate recall of a patient's entire clinical care and medical history. In controlled environments, such as primary care settings, the EHR has tremendous value. However in acute, uncontrolled, and non-traditional environments'e.g., surgery, rural/remote settings, emergency/trauma departments, and battlefields—the EHR is constrained due to its limited flexibility, non-intuitive work flows, menu-driven charting, reliance on robust communication connections, and dependence on manual data entry. Instead of aiding care, the EHR becomes a handicap, limiting the clinician's ability to provide hands-on clinical care. As such, there is an urgent need within healthcare settings to improve the interface and reduce the amount of time that clinicians spend interacting with the EHR; this is necessary to increase direct patient engagement and improve treatment outcomes.

Current practices in providing tactical field care and completing a Tactical Combat Casualty Care (TCCC) card affixed to a patient require that a lead medic provides care while a second acts as a scribe; recording information while following treatment guidelines. In this scenario, the lead medic may be distracted while communicating with his counterpart, while the second medic's skills are underutilized. Furthermore, documentation is at risk of error and loss during transfer to the military's MC4 electronic health record system. To improve military combat scenarios, there is a need to: 1) Reduce the number of medics per patient through hands-free, single-user data entry; 2) Incorporate an efficient data recording method to capture accurate information with reduced chance of human error; 3) Provide a streamlined solution that provides EHR continuity across disconnected groups, through digitally linking TCCC data to the MC4, with a robust solution for areas lacking internet connectivity.

Similar to the military environment, civilian first responders are typically disconnected from the local hospitals that receive their patients. In an emergency care situation, the first medical personnel to come into contact with a patient will initiate treatment; this includes an emergency medical technician (EMT), fire rescue, or emergency staff on presentation to an emergency department. Patient stabilization is the priority in these initial minutes with any care-related data being captured by whatever means available (often by writing on the backside of a latex glove). As the EHR is incompatible in acute/uncontrolled/non-traditional environments, documentation is often performed after the patient is stabilized, with clinicians relying on hand-written notes, verbal dictation, or memory to transfer information into the patient's EHR.

In larger mass casualty scenarios, limitations with the EHR are compounded. Due to the inability to log, treat, and track numerous patients that present in mass casualty events, patients are labeled with paper Triage Tags, color-coded in black, red, yellow and green, to signify one's degree of injury. These tags include space for writing pertinent medical information and serve as the primary means of field care documentation, and communication and information transfer between the field and the hospital. Similar to the TCCC in medical scenarios, noted limitations of current medical tags for civilian use include: 1) Limited space for recording medical data; 2) A format that allows only unidirectional changes in patient condition (worsening); 3) Tags that are not weather resistant, and are easily marred or destroyed; 4) A static and disconnected information repository, when real-time information regarding victims and their status is critical to the continuity of field care management.

TCCC cards are currently used to document patient condition and treatment of the patient prior to the patient arriving at the medical care facility. This data is entered onto the TCCC card with a pen and must be manually entered into the patients EHR upon arrival at the care facility. Additionally current practice is for the nurse/doctor to verbally interrogate the EMT/Medic upon arrival with the patient at the care facility. There is a need to streamline the documentation and communication of medical treatment performed early in emergency care situations, to capture treatment or condition data in real time with accurate time stamps, and to communicate that information to the team of clinicians in a timely and effective manner. Electronic medical data entry systems are also currently in use for military applications. One system utilizes a pen based user input method with a structured menu based interface. The pen-based input allows the user to input the data into the system, but requires the user to use their hands to enter the data. Data entry in this method is slow, tedious, and prone to errors. There exists a need to capture patient information in a way, which does not rely on pen, or touch based input methods.

### SUMMARY OF THE DISCLOSURE

This disclosure provides a hands-free solution to improve the interface between clinician providers and the EHR. The described systems and methods can include the following core features: flexibility in data entry methods allowing both structured list or check box based data entry and flexible context aware data entry; robust functioning in acute/uncontrolled/non-traditional environments such as emergency departments (ED) or battlefield care situations; the ability to provide EHR continuity across disconnected groups of care providers where different EHR systems are used to document the care of the same patient, such as when a patient is transferred from the emergency department of one hospital to another, or being transferred from a field aid station to a military hospital away from the front lines.

Disclosed herein is a method of documenting a medical condition of a patient, comprising the steps of evaluating the patient, inputting patient information into a personal computing device, and transmitting the patient information from the personal computing device to an electronic tag worn by the patient.

In some examples, the personal computing device can be a head-mounted display (HMD), a smartphone, or a smart watch.

The patient information can be inputted in a number of ways, including verbally inputting the patient information into the personal computing device, inputting the patient information with hand gestures, tracking an eye gaze of a user of the personal computing device, or inputting the patient information with a tradition input device such as a keyboard, mouse, or touchscreen.

In some examples, the method includes displaying a menu-based treatment checklist on the personal computing device, or displaying a menu based treatment checklist on the electronic tag.

The electronic tag can include electronics including a processor, memory, a battery, and a display (including a touch screen display). In some examples, the method can further include displaying the patient information on the electronic tag.

Additionally, the method can include providing treatment guidance with the personal computing device or the electronic tag. For example, treatment commands or prompts can be provided to the user through the personal computing device (e.g., voice or visual commands on an HMD) or through the electronic tag (e.g., on a display of the tag, or verbal commands through a speaker of the tag).

Also described herein is a method of documenting a medical condition of a patient, comprising the steps of transmitting patient information from an electronic tag worn by the patient into a personal computing device, evaluating the patient, updating the patient information in the personal computing device, and transmitting the updated patient information from the personal computing device to the electronic tag worn by the patient.

In some examples, the personal computing device can be a head-mounted display (HMD), a smartphone, or a smart watch.

The patient information can be inputted in a number of ways, including verbally inputting the patient information into the personal computing device, inputting the patient information with hand gestures, tracking an eye gaze of a user of the personal computing device, or inputting the patient information with a tradition input device such as a keyboard, mouse, or touchscreen.

In some examples, the method includes displaying a menu-based treatment checklist on the personal computing device, or displaying a menu based treatment checklist on the electronic tag.

The electronic tag can include electronics including a processor, memory, a battery, and a display (including a touch screen display). In some examples, the method can further include displaying the patient information on the electronic tag.

Additionally, the method can include providing treatment guidance with the personal computing device or the electronic tag. For example, treatment commands or prompts can be provided to the user through the personal computing device (e.g., voice or visual commands on an HMD) or through the electronic tag (e.g., on a display of the tag, or verbal commands through a speaker of the tag).

A patient care system is also provided, comprising a head-mounted display (HMD) comprising, a frame adapted to be worn on a head of a user, a camera disposed on or in the frame and configured to capture a digital image, a display disposed on or in the frame and configured to display the digital image to the user, a processor disposed on or in the frame and configured to control operation of the camera and the display, a non-transitory computer-readable storage medium disposed on or in the frame and configured to store a set of instructions executable by the processor; and an energy source disposed on or in the frame and configured to provide power to the camera, the display, the processor, and the non-transitory computer-readable storage medium, an electronic tag adapted to be worn by a patient, wherein the processor of the HMD is configured to receive patient information as an input from the user, transmit the patient information to the electronic tag, and receive the patient information from the electronic tag.

The electronic tag can include electronics including a processor, memory, a battery, and a display (including a touch screen display). In some examples, the electronic tag is configured to display patient information, or to sound an audible alarm when medical care is necessary.

In one example, the processor is further configured to provide treatment guidance through the display of the HMD.

An electronic medical tag adapted to be worn by a patient is also provided, comprising a housing, a processor disposed in the housing, a non-transitory computer-readable storage medium disposed in the housing and configured to store a set of instructions executable by the processor, a microphone, a wireless communication chip disposed in the housing, and an energy source disposed on or in the housing and configured to provide power to the processor, the non-transitory computer-readable storage medium, the microphone, and the wireless communication chip, wherein the processor is configured to control the electronic medical tag to receive patient information as an input from a user with the microphone, store the patient information in the non-transitory computer-readable storage medium, and transmit the patient information to a remote electronic device with the wireless communication chip.

The electronic tag can include electronics including a processor, memory, a battery, and a display (including a touch screen display). In some examples, the electronic tag is configured to display patient information, or to sound an audible alarm when medical care is necessary.

In one example, the processor is further configured to provide treatment guidance through the display or a speaker of the electronic tag.

A method of documenting a medical condition of a patient is further provided, comprising the steps of evaluating the patient, verbally inputting patient information into an electronic tag worn by the patient, and transmitting the patient information from the electronic tag to a separate electronic device.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an example of a head mounted display (HMD) 100 which incorporates a variety of sensors, data input methods, data display methods, and networking to record data.

FIGS. 2A-2B illustrate one embodiment of a HMD.

FIG. 3 illustrates an electronic TCCC tag (E-TC3) or data tag.

FIG. 4 illustrates communication between a HMD, add-on modules (microphone, processor, battery, range finder, and/or camera) and the E-TC3 tag.

FIG. 5 is a schematic of the components incorporated into the data tag.

FIG. 6 is a schematic of the components incorporated into a standalone data tag.

FIG. 7 is a method of providing and documenting care of a patient using a HMD and a data tag configured to record the patients treatment data.

FIG. 8 illustrates a software architecture for collecting and reviewing patient information by and on the HMD.

### DETAILED DESCRIPTION

It is to be further understood that the present disclosure is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present disclosure. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Features, which are described in the context of separate embodiments, may also be provided in combination in a single embodiment. Conversely, various features, which are, for brevity, described in the context of a single embodiment, may also be provided separately or in any suitable sub-combination. The Applicants hereby give notice that new claims may be formulated to such features and/or combinations of such features during the prosecution of the present Application or of any further Application derived therefrom.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

A head mounted display (HMD) may refer to one or more apparatus and/or one or more systems that are capable of accepting input from the user via a variety of input methods. Touch, voice, head tilt/motion, eye tracking are all examples of input methods into HMD systems. A head mounted display integrates visual display of images and text to the user, a microprocessor capable of executing instructions via software programs also known as apps or app. A head mounted display may also include computer memory, a digital camera, a motion sensor, and communicate with networks via wireless communication protocols.

“Software” may refer to prescribed rules to operate a computer. Examples of software may include: code segments in one or more computer-readable languages; graphical and or/textual instructions; applets; pre-compiled code; interpreted code; compiled code; and computer programs.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash memory; a memory chip; and/or other types of media that can store machine-readable instructions thereon. Non-volatile storage is a type of computer readable medium which does not lose the information stored inside when power is removed from the storage medium.

A “computer system” may refer to a system having one or more computers, where each computer may include computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

Exemplary networks may operate with any of a number of protocols, such as Internet protocol (IP), asynchronous transfer mode (ATM), and/or synchronous optical network (SONET), user datagram protocol (UDP), IEEE 802.x. Bluetooth is an example or an IEEE standard under IEEE 802.15.1.

Embodiments of the present disclosure may include apparatuses for performing the operations disclosed herein. An apparatus may be specially constructed for the desired purposes, or it may comprise a general-purpose device selectively activated or reconfigured by a program stored in the device.

Embodiments of the disclosure may also be implemented in one or a combination of hardware, firmware, and software. They may also be implemented as instructions stored on a machine-readable medium, which may be read and executed by a computing platform to perform the operations described herein.

The term user, operator, physician, nurse, EMT, medic, or clinician refers to the person delivering care to a patient. The term patient, casualty, accident victim, or injured refers to the patient receiving care.

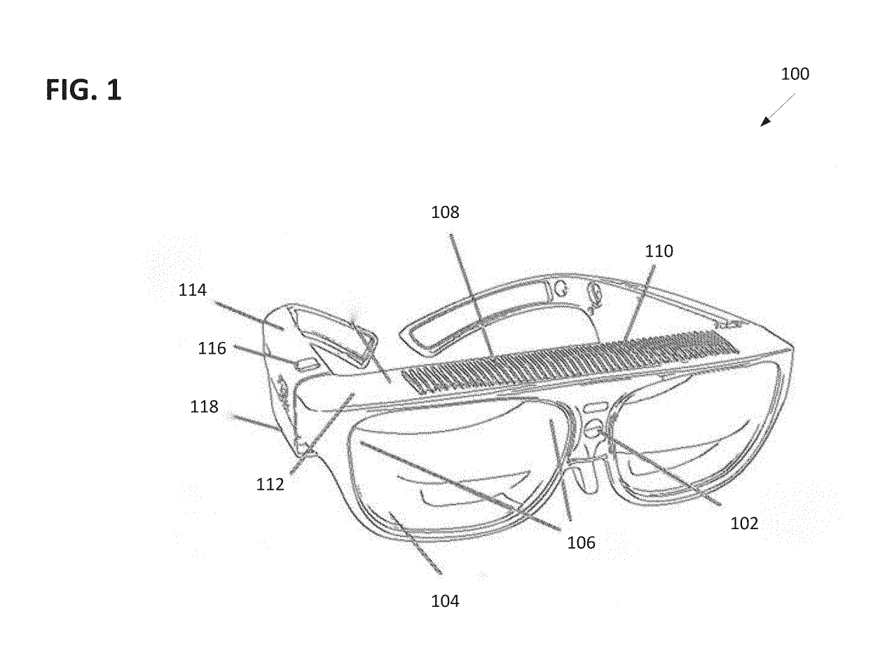

FIG. 1 is an example of a head mounted display (HMD) 100 which incorporates a variety of sensors, data input methods, data display methods, and networking to record data. For example, the HMD can include a camera 102, lenses 104, magnetic lens connectors 106, and a frame 108 that houses or supports a processor 110, wireless chip 112 (such as Bluetooth or WiFi), batteries 114, a trackpad 116, control buttons 118, and sensors 120 (such as accelerometers, gyroscopes, magnetometers, altitude sensors, humidity sensors, etc.). In some examples, the HMD can be integrated into a helmet or head gear (to be worn on a battlefield).

This head mounted display is capable of receiving input through a microphone and responds to voice commands. The microphone is configured to incorporate noise-cancelling techniques to provide a noise reduced voice signal to the voice to text processor in the HMD and additional hardware. This microphone can be configured to be of a boom style, and/or may be configured to be noise cancelling, where an ambient microphone records the ambient noise and outputs an inverted noise signal into the boom microphone, reducing the perceived loudness of the noise while boosting the clarity of the voice signal. To improve the performance of the speech recognizer, a boom microphone may be implemented. If the distance from a user's mouth to the HMD's built-in microphone is 100 mm, a microphone mounted on a boom that extends to the front of the speaker's mouth will reduce the distance to 10 mm. Because the sound intensity from a point source of sound will obey the inverse square law if there are no reflections or reverberation, the intensity of the speech signal will theoretically be 20 dB higher, leading to a considerable improvement in signal-to-noise ratio (SNR).

The HMD also configured to be controlled via touch/trackpad/button commands. The HMD is capable of: performing on-board processing the data from the voice commands, displaying a menu based treatment checklist, broadcasting audio output, and transmitting patient data via network protocols. The accelerometer, gyroscope, magnetometer, altitude sensor, and humidity sensors are able to record data relating to patient treatment. Additionally the HMD is configured to provide a digital clock or chronometer to record the time of treatment. The HMD is also configured to include an auto-focus camera for recording photographic and video images of the patient during treatment. The HMD incorporates a microprocessor with onboard RAM, flash non-volatile storage, and runs an operating system.

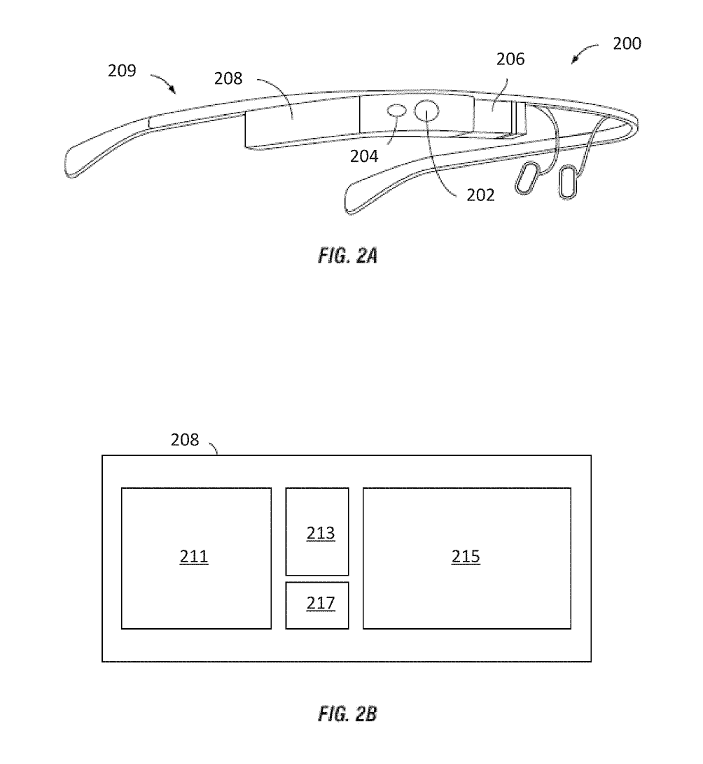

FIGS. 2A-2B illustrate one embodiment of a HMD 200, that can include a camera 202, additional sensor(s) 204, display 206, electronics compartment 208, and frame 209. In this embodiment, the frame comprises glasses frames and can be worn on the head of a user and be supported by the user's ears and nose. As shown in FIG. 2B, the electronics compartment can house a processor 211, a non-transitory computer-readable storage medium 213 configured to store a set of instructions capable of being executed by the processor, and an energy source 215 such as a battery to power the device. The electronics compartment can also include additional electronics 217 which can be a microphone, wireless communications electronics such as WiFi, cellular, or Bluetooth chips that enable the wound assessment device to communicate with other devices and computers wirelessly, imaging processing microchips, gyroscopic position and orientation sensors, eye tracking sensors, eye blink sensors, touch sensitive sensors, speakers, vibratory haptic feedback transducers, stereoscopic cameras, or other similar electronics and hardware typically found on smartphones and digital devices. While the HMD 200 is illustrated as a hands-free, wearable device, in other embodiments the wound assessment device can be a smartphone, PC, tablet, or other electronic device that includes the components described above including a camera, a processor, non-transitory computer-readable storage medium, a display, and an energy source.

The processor 211 can be configured to control the operation of the wound assessment device, including executing instructions and/or computer code stored on the non-transitory computer-readable storage medium 213, processing data captured by the camera 202 and additional sensor(s) 204, and presenting information to the display 206 for display to the user of the device. In some embodiments, the processor is configured to determine the dimensions of the wound and to overlay a digital ruler or measurement scale on top of digital images of the wound for documentation purposes. In some embodiments, the processor can determine the dimensions of the wound without requiring a physical measurement device or reference marker to be positioned on or near the wound. The modified image with the overlaid digital ruler or measurement scale can be stored on the non-transitory computer-readable storage medium 213, displayed on the display 206, stored in the patient's electronic medical record, and/or transmitted to another computer or device for storage, display, or further manipulation or study.

The processor can further be configured to affix or overlay patient information such as name, date of birth, and other identifying information from the patient or the patient's chart onto the display. This information can be acquired automatically by the processor from an electronic medical tag, can be entered manually by the user, or can be verbally spoken into the microphone of the HMD and processed with speech recognition software. Additionally, the processor 211 may be configured to offload processor intensive operations to an additional computer, mobile phone, or tablet via the wireless connections such as WiFi, cellular, or Bluetooth.

The camera 202 can be configured to capture digital images and/or high-resolution video which can be processed by the processor 211 and stored by the non-transitory computer readable storage medium 213, or alternatively, can be transmitted to a separate device for storage. The camera can include a zoom lens or a fixed focal length lens, and can include adjustable or auto-focus capabilities or have a fixed focus. In some embodiments, the camera can be controlled to take images/video by pressing a button, either on the HMD itself or on a separate device (such as a smartphone, PC, or tablet). In other embodiments, the user can use voice control to take images/video by speaking into the microphone of the wound assessment device, which can process the command with speech recognition software to activate the camera. In one embodiment, the camera 202 may be a stereoscopic camera with more than one lens which can take simultaneous images of the patient at a known camera angle between the cameras focusing on the same point of the image. The stereoscopic images along with the camera angle can be used to create a three dimensional image of the patient.

The additional sensor(s) 204 can include an infra-red sensor, optical sensor, ultrasound sensor, acoustic sensor, a laser, a thermal sensor, gyroscopic position and orientation sensors, eye tracking sensors, eye blink sensors, touch sensitive sensors, speakers, vibratory haptic feedback transducers, stereoscopic cameras, or the like. The additional sensor(s) can be used to provide additional information to the processor for processing image data from the camera.

The display 206 can be a see-through display that allows a user to see through the display but also view what is being shown on the display by the HMD. The display can be, for example, an OLED screen with multiple layers of glass or transparent material surrounding the OLED. While the wound assessment device 200 of FIG. 2A includes a single display 106 in front of only one eye of the user, it should be understood that in other embodiments, the wound assessment device can include two displays (one in front of each eye of the user) or a single large display that extends across the periphery of both eyes of the user.

The HMDs of described herein can be a version of a wearable computer, which is worn on the head and features a display in front of one or both eyes. The HMD is configured to provide a portable, hands-free environment. The environment of the HMD is configured to provide a user to computer interface. The preferred embodiment of the computer interface is a hands-free interface to allow caregivers to provide care with their hands while the computer interface displays information to the caregiver and/or the caregiver records patient data. Types of hands-free interfaces include voice-based, eye-based, electromyographic (EMG)-based, gesture-based, and electroencephalographic (EEG)-based.

The HMDs of described herein can be configured to have a voice-based user interface (VUI): Voice user interfaces are uniquely based on spoken language, learned implicitly at a young age, whereas other user interfaces depend on specific learned actions designed to accomplish a task, such as selecting an item from a drop-down menu or dragging and dropping icons. The performance of the VUI is naturally dependent on accurate speech-recognition software, described below.

The HMDs of described herein can be configured to have an eye-based user interface: Tracking eye gaze as a form of control was primarily developed for people unable to operate a keyboard and pointing device. Small cameras incorporated into an HMD observe user pupils, while calibrated software calculates gaze direction.

The HMDs of described herein can be configured to have an electromyographic (EMG)-based user interface: EMG-based control translates the electrical signals associated with muscle contractions into control inputs. For instance, the Myo band (Thalmic Labs, Kitchener, Ontario) measures EMG signals in the muscles of a user's forearm as she makes different hand and arm gestures. Myo also has an inertial measurement unit (IMU) comprising a 3-axis gyro, accelerometer, and magnetometer. One application of Myo is as a computer mice replacement for pointing and clicking.

The HMDs of described herein can be configured to have a gesture-based user interface: In contrast to EMG-based methods, gesture-based control tracks the displacements of a body part, such as the head, eye brow, jaw, or (most commonly) the hand. Detecting hand gestures has traditionally required usage of an additional device, such as a glove or wrist band. GestureWrist was an early wristwatch-type input device that recognized human hand gestures by measuring accelerations with an accelerometer and changes in wrist shape through capacitive sensing. Recent advances have allowed hand movement to be tracked visually via cameras, leaving the hands unencumbered. Although hand-gesture-based interfaces are not practical in hands-busy applications, they could be useful before and after an intervention.

The HMDs of described herein can be configured to have an electroencephalographic (EEG)-based user interface: Researchers are pushing the limits of human input by allowing users to control computers with their thoughts. “Brain caps,” fitted with non-invasive EEG sensors that record brain activity, are tethered to computers with fast processors that analyze the signals in real time.

The hands-free user interface methods described so far are most applicable for executing specified commands. When inputting unstructured information in a hands-free manner, a different type of interface is preferred. One-handed keyboards, handwriting recognition systems, and gesture-to-text programs are not truly hands-free and are not preferred. The HMDs described herein preferably are configured to have a speech recognition capability, providing a user-friendly, unobtrusive, flexible and efficient method of inputting unstructured information. Speech recognition performance is traditionally reported as the word error rate (WER), defined as the edit distance between the reference word sequence and the sequence emitted by the transcriber. WER=(S+D+I)/N, where S, D, and I are the number of substitutions, deletions, and insertions, respectively, and N is the number of words in the reference. Two primary sources for increased word error rates are noisy environments and speaker particularities, such as accents. To provide accurate voice to text translation and to reduce the WER, the HMDs described herein can be configured with an add-on module of an external microphone.

The HMDs described herein can be configured to employ traditional speech recognition systems, which are based on a hidden Markov model (HMM) in which each state is modeled by a Gaussian mixture model (GMM). The HMDs described herein can also be configured to employ an acoustic model based on a deep neural network (DNN) has led to significant improvements over GMM-based systems. State-of-the-art systems are now using long short-term memory (LSTM), a type of recurring neural network (RNN) trained with connectionist temporal classification (CTC). The speech recognition systems have become much more robust to noise, reducing the WER by 20-40% over the past years.

To varying degrees, hands-free methods have been used to interact with EHRs. Speech-based control and speech recognition technology are by far the most mature technologies, though their adoption in the medical domain has been slow. Researchers who conducted a survey at Vejle and Give Hospital—one of the first hospitals in Denmark to introduce speech recognition technology in all departments—found that 33% of physicians agreed that speech recognition technology was a good idea, 31% did not, and 36% were neutral [16]. The software used was Philips Speech Magic, adapted to Danish. Eight years later, another group replicated the study at Mercy Health using DNN-based Nuance Dragon in English, reporting that 87% of physicians agreed that speech recognition technology was a good idea and 51% of physicians reported time savings.

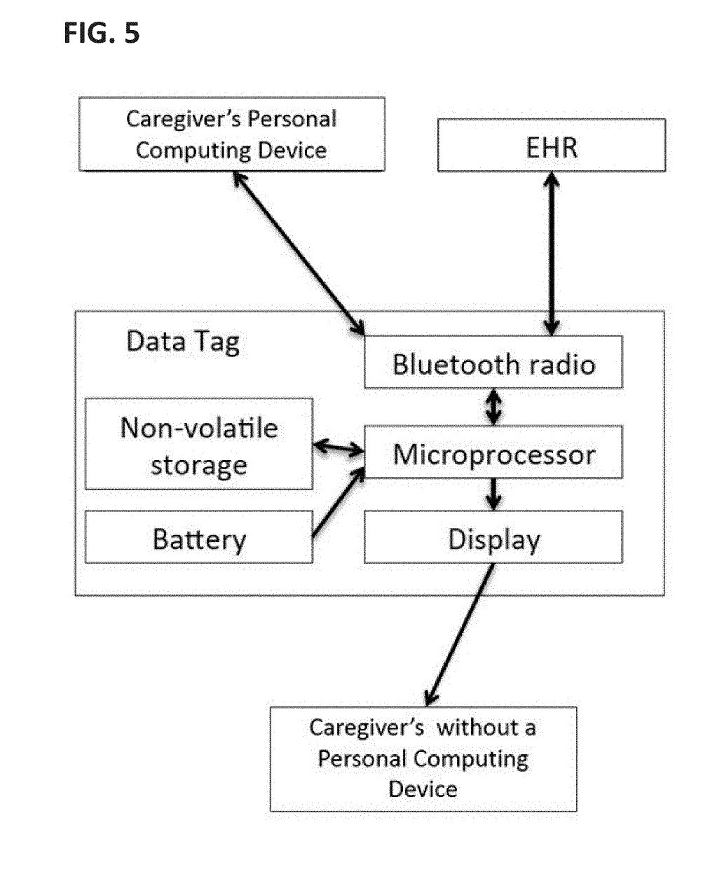

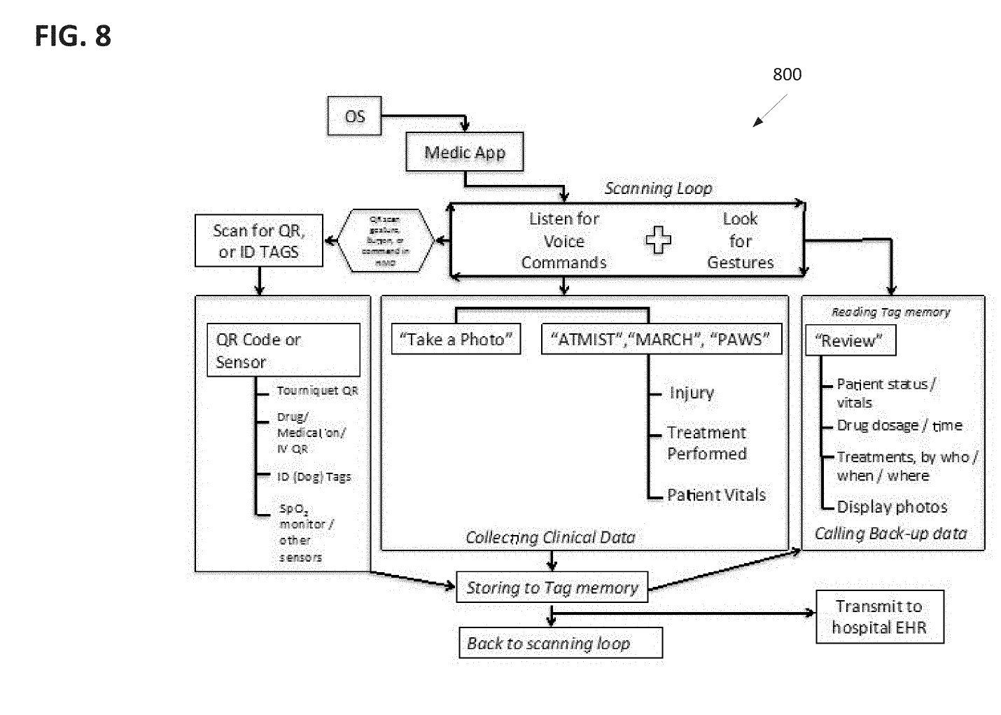

The HMDs described herein may further be configured to incorporate an automatic speech recognition (ASR) system. The ASR on a mobile/wearable processor would run continuously, provide a low-latency response, have a large vocabulary, and operate with minimal battery drain. The system of FIG. 8 is further configured to incorporate Deep Neural Network (DNN) support in the ASR to improve speech recognition. The ASR of FIG. 5 further is configured to have a customized language model specific to medical, EMT, and/or military application environments.

The HMDs described herein are further configured to include software and hardware capable of reading patient information off of a patient wrist band or patient identification card. Bar-code scanning, optical character recognition (OCR), radio frequency identification (RFID), 2-d barcode, or other data entry methods may be employed. An example of OCR data entry is the automatic reading of a patients name or other information off of a military identification tag.

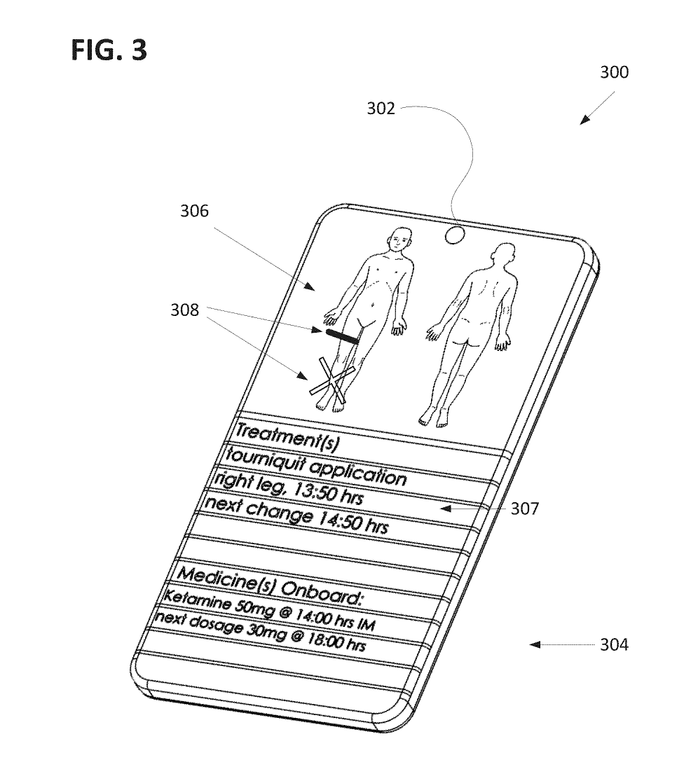

FIG. 3 is an electronic TCCC tag (E-TC3 ) 300 or data tag, which is configured to be attached to the patient at the time of treatment by the caregiver. The tag may be affixed to the patient with: a lanyard, a strap, a wrist or ankle band, adhesive, an armband, tape, hook and loop fasteners, safety pins, buttons, snaps, or other methods. A hole 302 for affixing a lanyard is shown in FIG. 3. Other affixing methods may be employed on the back or sides of the tag.

The tag further comprises electronics 304, including a network link to communicate with a HMD of the present disclosure. The HMD is the interface between the user and the data tag. The electronics 304 of the data tag can further include a data storage microchip, a microprocessor, and a battery. The data transmitted to the data tag from the HMD can be stored in the data tag on internal non-volatile storage such as flash memory, hard drive, or other non-volatile memory methods.

The data tag is further configured to contain a display 306, which will display selected patient information on the external surfaces of the data tag. The display is constructed as an LCD display however LED, oled, or e-ink style displays may be used. In some embodiments, the display covers the entire front side of the electronic tag, similar to a smartphone display. The display may be monochrome, or full color, or a combination of each. The display may include a touch screen interface for scrolling or changing pages to display more patient information. The display of this information is to inform clinicians, transportation EMT's or other caregivers who are not wearing a HMD. The patient's vital signs, triage status, injury location, treatments given, drugs or other medications administered, time of drug administration, tourniquets applied, time of tourniquet application, and or time of next tourniquet change and/or loosening are selectively displayed in text 307 so the caregivers have the critical patient information clearly and easily at hand. The tag may also display patient allergies, drug combination errors, and/or clinical decision support recommendations. For example, referring to FIG. 3, the display 306 shows an image of a patient and user added markers 308, including an “X” that marks an untreated injury on the lower leg, and further displays the application of a tourniquet on the upper leg of the patient. These user added markers 308 can be added to and removed from the electronic tag in real time by the caregiver (such as a medic on the battlefield) to keep track of patient treatment.

The tag may also be equipped with a timer and a speaker to provide an audible alarm to alert caregivers of clinical care which is required at a certain time. For example, such an audible alarm would be useful to alert caregivers that a tourniquet needs to be adjusted within a certain period of time after tourniquet application. The tag may be configured to be a function of a smartwatch which is pre-worn by the patient. At the time of care, the personal computing device of the caregiver connects with the tag and records patient treatment information.

The data tag of FIG. 3 is configured to include a battery for powering the functionality described above. The battery can be sized such that the data tag is powered for years at a time. Alternatively, as battery power is critical to the function of the device, the tag of FIG. 3 can be configured to have a battery installed where the battery is not drained during storage. Upon affixing of the data tag to the patient a switch is flipped or an insulating film is released from the battery contacts to permit the battery to power the data tag. The tag is then paired with the HMD via Bluetooth, WiFi direct, or other communication protocols, and data storage and display may commence. As treatment occurs, treatment data is recorded by the HMD and stored on the data tag. Alternatively, the tag may include a microphone and touchscreen interface to collect data from the treatment of the patient without the use of an HMD. Such a tag is disclosed in FIG. 11.

Once the patient is stable the patient is transported to a care facility such as a hospital, field aid station, or other fixed medical facility. At that facility, there is a reader configured to read the data off of the data tag and incorporate the patient's medical information stored on the tag into the hospital's electronic health record system (EHR). Once the data is read from the data tag, the data tag can be configured to destroy the data inside to protect patient privacy.

The data tag of FIG. 3 is configured to be disposed of after the patient is transferred to a care facility.

The visual displays of the HMDs described herein are configured to provide the user with an augmented reality computer environment where menu commands are displayed on the inside of the lenses of the glasses. The menu system can be configured to be activated by voice commands, touch, or button commands. The menu system is configured to provide a treatment checklist to the user for treatment of the patient. The treatment checklist is stepped through by the user who is administering care with both hands, while the HMD is providing treatment information to the user and recording patient information via voice commands by the user. The patient information is then transmitted to the tag of FIG. 3.

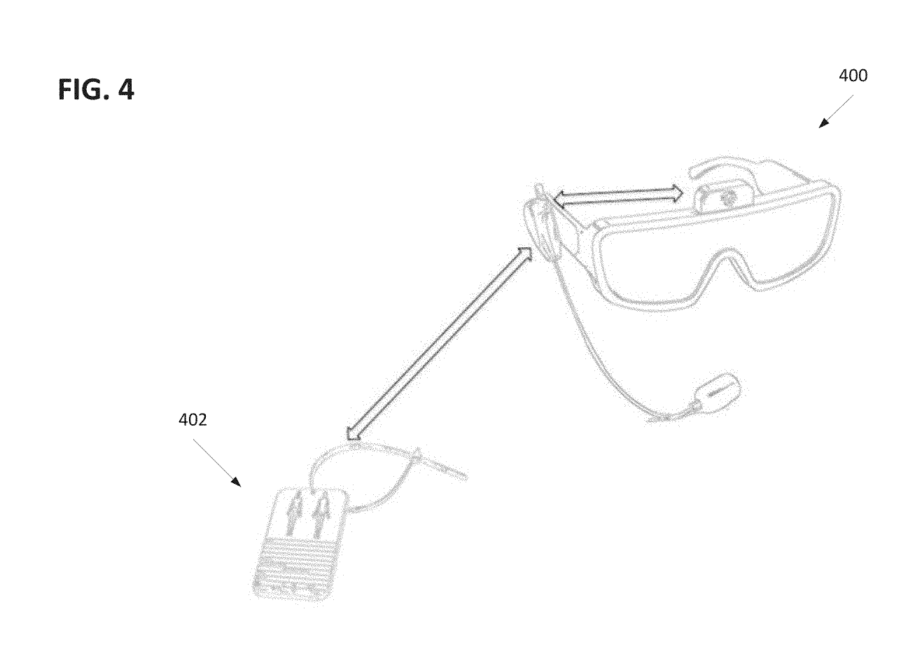

FIG. 4 illustrations communication between a HMD 400, add-on modules (microphone, processor, battery, range finder, and/or camera) and the E-TC3 tag 402 or data tag. In some embodiments, the E-TC3 tag can be a smartphone. The E-TC3 tag of FIG. 3 is also capable of communicating with the HMD via a wireless communications protocol. Alternatively, a wired communication method could be used for either or all of the communication pathways shown in FIG. 4. The wireless communication protocol shown is Bluetooth low energy (BLE) but any Bluetooth, WiFi, LTE, satellite, or other communication method could be employed. A Pre-Hospital Treatment & Triage (PHT) treatment guidance system, visible in the HMD, will guide caregivers and/or first responders through treating the patient and documenting a patient's medical condition and treatment status, and triaging patients to the appropriate level of care.

The system shown in FIG. 4 is configured to guide the caregiver through a pre-hospital treatment and triage checklist, automatically transcribe the data, and remotely store the information on the E-TC3 tag. Based on the patient's health status, the physical E-TC3 tag will turn black, red, yellow or green, to indicate transport to the appropriate next-level care facility. Transcription and storage of the patient's care in the field would allow for a complete account of all treatment provided and a detailed time stamp of such events, thereby creating continuity in the EHR through capturing the first (“golden hour”) of treatment.

Upon arrival at the hospital or other care setting, clinical staff would access the patient's medical data and all relevant demographic information (digitally captured in the E-TC3 ), and securely upload the data to the patient's EHR. This upload would occur automatically via a HIPAA-compliant Bluetooth connection, without the need for a clinician to manually enter data or for internet connectivity. After use, the disposable E-TC3 would erase all protected health information (PHI) onboard to protect confidential patient data.

As emergency medical procedures are becoming more portable and mobile, the system shown in FIG. 4 provides an opportunity to perform more complex critical procedures closer to the initial point of engagement with an EMT or field medic. As health care attempts to provide more value, by providing better outcomes for the same or less cost, our HMD and E-TC3 system significantly shifts the value equation by reducing the need for “scribes,” increasing safety through removing asynchronous charting, and elevating the clinical practice of personnel

The system of FIG. 4 further may incorporate the ability to communicate and integrate PHI from additional smart medical devices such as existing BLE-enabled devices (such as pulse oximeters, thermometers, and blood pressure cuffs) to automatically populate the E-TC3 tag with digital vital sign data, upon first presentation of injury in the field. The system of FIG. 4 further may incorporate the ability to input data through Optical Character Recognition (OCR) to scan a military ID tag data rapidly and incorporate this information into the E-TC3 digital tag (to accompany barcode scanning for civilian use). The system of FIG. 4 further may provide EHR Integration: Ability for the E-TC3 to communicate with the military's Medical Communications for Combat Casualty Care (MC4) system, and to connect with civilian EHR systems via BLE connectivity.

FIG. 5 is a schematic of the components incorporated into the data tag. The tag is configured to include a microprocessor to manage data flows. A Bluetooth radio communicates with the HMD to send and receive data to and from the tag. The radio also sends and/or receives data from the hospital's EHR once the patient and the tag are transported to the hospital. The tag is configured to include non-volatile storage for recording the patient's health records. The tag is configured to include a low cost display for communicating health record to caregivers without HMD hardware. An internal battery powers the data tag. The HMD of the caregiver may also be replaced with a smartphone, smart watch, or other personal computer with the ability to receive patient information from the caregiver, communicate with the data tag, and/or display information to the caregiver.

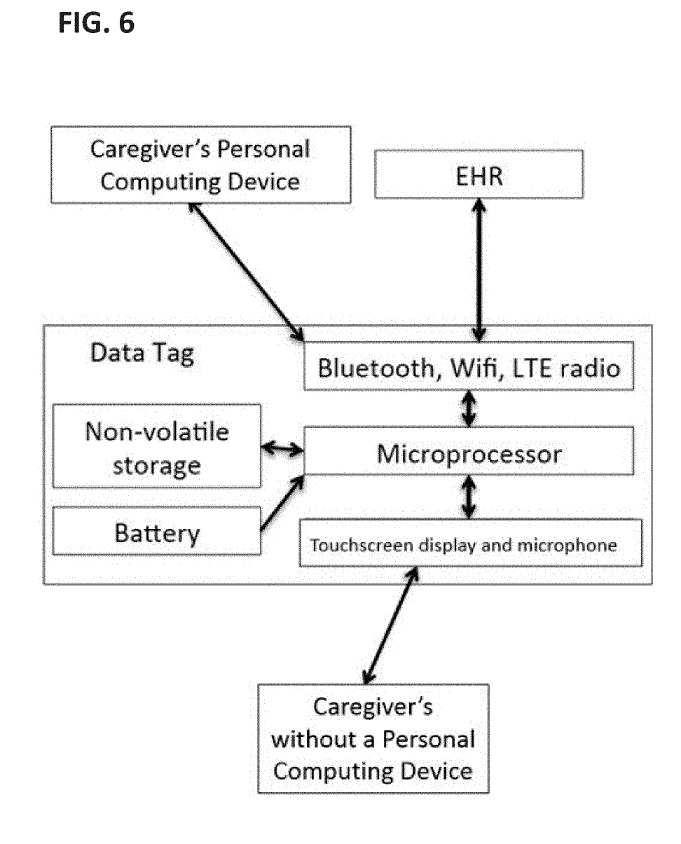

FIG. 6 is a schematic of the components incorporated into a standalone data tag. This tag is similar to the tag of FIG. 5 with the inclusion of a touch screen display and a microphone to allow caregivers without a HMD to provide data input into the data tag. The microphone includes noise canceling and or far field microphone array hardware to enable the tag to discern a care givers voice over background noise. These data input methods are configured to allow a caregiver who is a non-traditional or non-trained medical caregiver to provide care or treatment. The data tag is configured to offer care instructions and offer a touch screen checklist for the inexperienced caregiver to follow to administer care and record that care on the data tag.

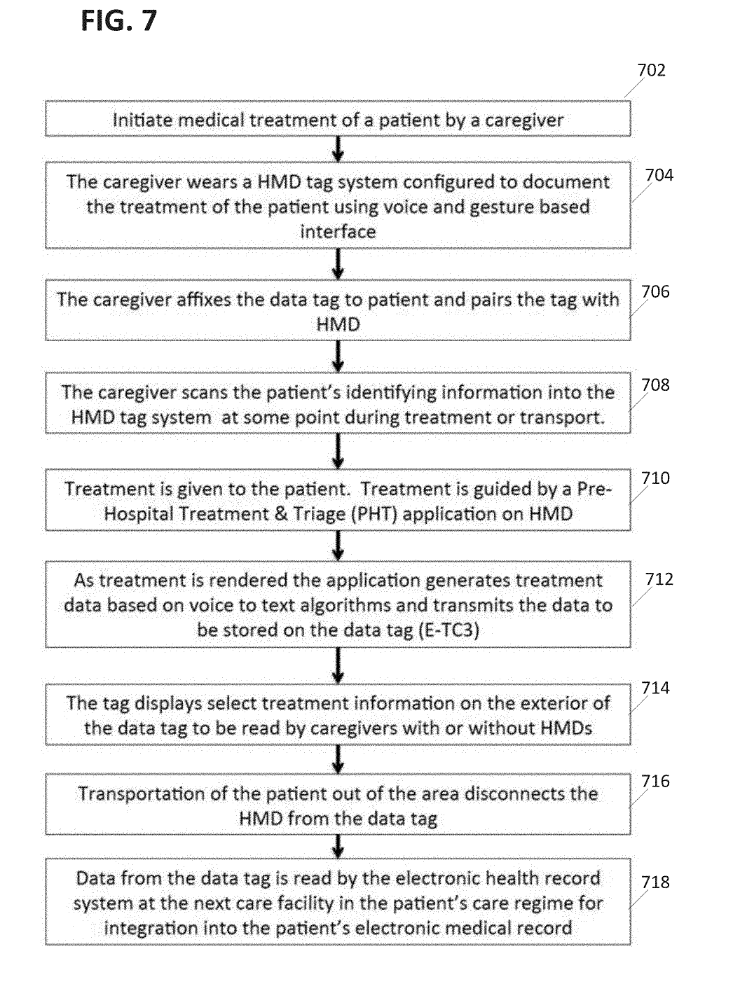

FIG. 7 is a method of providing and documenting care of a patient using a HMD and a data tag configured to record the patients treatment data. The HMD is configured to include voice commands to allow the caregiver to provide a method of treatment with both hands while voice to text processing in the HMD records the patient's treatment on the data tag. At step 702 of the method of FIG. 7, the method comprises initiating medical treatment of a patient by a caregiver. At step 704 of FIG. 7, the method comprises providing the caregiver with a HMD tag system configured to document the treatment of the patient using a voice and gesture based interface. At step 706, the caregiver affixes the data tag to the patient and pairs the tag with the HMD. At step 708, the caregiver scans the patient's identifying information into the HMD tag system at some point during treatment or transport. At step 710, treatment is given to the patient. Treatment is guided by a pre-hospital treatment and triage (PHT) application on the HMD. At step 712, as treatment is rendered, the application generates treatment data based on voice to text algorithms and transmits the data to be stored on the data tag. At step 714, the tag displays select treatment information on the exterior of the data tag to be read by caregivers with or without HMDs. At step 716, the patient is transported out of the area disconnecting the HMD from the data tag. At step 718, data from the data tag is read by the electronic health record system at the next care facility in the patient's care regime for integration into the patient's electronic medical record.

FIG. 8 illustrates a software architecture 800 for collecting and reviewing patient information by and on the HMD. The operating system of the HMD ‘OS” runs the “Medic app”. The medic app utilizes the hardware capabilities of the HMD allow the operator to place the HMD into a continually scanning mode where the microphone in conjunction with gesture recognition and augmented by barcode or qr code recognition and/or object recognition will scan the immediate area for medical treatments happening. Once a treatment is identified, the HMD will categorize that treatment and write the specific details of the treatment to the tag. This collecting of clinical data can be structured to follow a clinical triage/treatment scenario. Such scenarios may be represented by acronyms such as “ATMIST”, “PAWS”, or “MARCH”. ATMIST stands for recording patient: Age, Time of incident, Mechanism of Injury, Injuries, Vital Signs, Treatment Given. March stands for evaluating patient: M—Massive Bleeding. A—Airway. R—Respirations. C—Circulation. H—Head. Other treatment guidelines such as PAWS, PEWS, or others may also be employed to guide treatment, record patient data, or score the patient for order of treatment.

The data recorded as part of these treatment scenarios may be displayed locally on the tag, transmitted to the caregivers HMD, or both. The data recorded is then transmitted to the hospital's electronic health record system (HER, such as the US military's MC4 system or others) at the time of admission of the patient into the hospital. Alternatively the HMD may take photographs for later transmission to colleagues away from the site of treatment. The HMD will take inventory of medications or medical devices used and time stamp the application of treatment/medication. The storing of the treatment data on the tag allows the data to be preserved until the patient is transported to a location where network connectivity is possible (such as a medivac helicopter, or ambulance). At such time, the clinical data collected may be transmitted to the hospital or treatment facility, ahead of the patient.

The data structures and code described in this detailed description are typically stored on a non-transitory computer-readable storage medium, which may be any device or medium that can store code and/or data for use by a computer system. The non-transitory computer-readable storage medium includes, but is not limited to, volatile memory, non-volatile memory, magnetic and optical storage devices such as disk drives, magnetic tape, CDs (compact discs), DVDs (digital versatile discs or digital video discs), or other media capable of storing computer-readable media now known or later developed.

The methods and processes described in the detailed description section can be embodied as code and/or data, which can be stored in a non-transitory computer-readable storage medium as described above. When a computer system reads and executes the code and/or data stored on the non-transitory computer-readable storage medium, the computer system performs the methods and processes embodied as data structures and code and stored within the computer-readable storage medium.

Furthermore, the methods and processes described above can be included in hardware modules. For example, the hardware modules can include, but are not limited to, application-specific integrated circuit (ASIC) chips, field-programmable gate arrays (FPGAs), and other programmable-logic devices now known or later developed. When the hardware modules are activated, the hardware modules perform the methods and processes included within the hardware modules.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of documenting a medical condition of a patient, comprising the steps of:
evaluating the patient;
inputting patient information into a personal computing device; and
transmitting the patient information from the personal computing device to an electronic tag worn by the patient.

2. The method of claim 1, wherein the personal computing device comprises a head-mounted display (HMD).

3. The method of claim 1, wherein the personal computing device comprises a smartphone.

4. The method of claim 1, wherein the personal computing device comprises a smart watch.

5. The method of claim 1, wherein the inputting patient information step comprises verbally inputting the patient information into the personal computing device.

6. The method of claim 2, wherein the inputting patient information step comprises inputting the patient information with hand gestures.

7. The method of claim 2, further comprising displaying a menu-based treatment checklist on the personal computing device.

8. The method of claim 1, further comprising displaying a menu based treatment checklist on the electronic tag.

9. The method of claim 2, wherein the inputting patient information step comprises tracking an eye gaze of a user of the personal computing device.

10. The method of claim 1, further comprising displaying the patient information on the electronic tag.

11. The method of claim 1, further comprising providing treatment guidance with the personal computing device or the electronic tag.

12. A patient care system, comprising:
a head-mounted display (HMD) comprising:
a frame adapted to be worn on a head of a user;
a camera disposed on or in the frame and configured to capture a digital image;
a display disposed on or in the frame and configured to display the digital image to the user;
a processor disposed on or in the frame and configured to control operation of the camera and the display;
a non-transitory computer-readable storage medium disposed on or in the frame and configured to store a set of instructions executable by the processor; and
an energy source disposed on or in the frame and configured to provide power to the camera, the display, the processor, and the non-transitory computer-readable storage medium;
an electronic tag adapted to be worn by a patient;
wherein the processor of the HMD is configured to receive patient information as an input from the user, transmit the patient information to the electronic tag, and receive the patient information from the electronic tag.

13. The system of claim 12 wherein the electronic tag is configured to display patient information.

14. The system of claim 12, wherein the electronic tag is configured to sound an audible alarm when medical care is necessary.

15. The system of claim 12, wherein the electronic tag further comprises a display, the display being configured to display the patient information.

16. The system of claim 12, wherein the processor is further configured to provide treatment guidance through the display of the HMD.

17. An electronic medical tag adapted to be worn by a patient, comprising:
a housing;
a processor disposed in the housing;
a non-transitory computer-readable storage medium disposed in the housing and configured to store a set of instructions executable by the processor;
a microphone;
a wireless communication chip disposed in the housing; and
an energy source disposed on or in the housing and configured to provide power to the processor, the non-transitory computer-readable storage medium, the microphone, and the wireless communication chip;
wherein the processor is configured to control the electronic medical tag to receive patient information as an input from a user with the microphone, store the patient information in the non-transitory computer-readable storage medium, and transmit the patient information to a remote electronic device with the wireless communication chip.

18. The system of claim 17, wherein the electronic medical tag further comprises a display to display the patient information.

19. The system of claim 17, wherein the electronic medical tag is configured to be affixed to the patient.

20. The system of claim 17, wherein the processor is configured to provide treatment guidance with the display.

21. The system of claim 17, further comprising a speaker.

22. The system of claim 21, wherein the processor is configured to provide treatment guidance with the speaker.

23. The system of claim 19, wherein the tag is a smartwatch.
